(19) United States
(12) Patent Application Publication
(10) Pub. No.: US 2024/0366936 A1
(43) Pub. Date: Nov. 7, 2024

(54) FUNCTIONAL ELECTRICAL STIMULATION (FES) APPLICATOR

(71) Applicant: Griffith University, Nathan (AU)

(72) Inventors: Adrian McCormack, Nathan (AU); Claudio Pizzolato, Nathan (AU); Sam Canning, Nathan (AU); Kyle Mulholland, Nathan (AU)

(21) Appl. No.: 18/686,330

(22) PCT Filed: Aug. 22, 2022
(86) PCT No.: PCT/AU2022/050943

§ 371 Date: Feb. 23, 2024

(30) Foreign Application Priority Data
Aug. 24, 2021 (AU) 2021106863

(51) Int. Cl. A61N 1/36; A61N 1/04

(52) U.S. Cl. CPC A61N 1/36014; A61N 1/0452; A61N 1/0456; A61N 1/0476; A61N 1/0484; A61N 1/36003

(57) ABSTRACT

The present invention relates to an applicator for applying functional electrical stimulation (FES) to rehabilitate a person. The applicator includes a surround for surrounding a portion of the person. One or more electrodes are locating within the surround. The electrodes may be borne by the surround, when attaching the surround to the portion, to greatly reduce set-up time.

### TECHNICAL FIELD

The present invention relates to a functional electrical stimulation (FES) applicator.

### BACKGROUND

The reference to any prior art in this specification is not, and should not be taken as an acknowledgement or any form of suggestion that the prior art forms part of the common general knowledge.

PCT/AU2020/050566 a rehabilitation system for rehabilitating a person with a neurological condition such as a spinal cord injury (SCI). The system generates functional electrical stimulation (FES) for application to the person.

FES interventions for SCI involves the application of low-level electrical impulses to nerves or muscles to improve and/or restore muscle function. The electrical impulses help muscles to contract, triggering the desired therapeutic outcome. FES devices consist of electrodes which are connected to a neuromuscular electrical stimulator unit. The electrodes are attached to the surface of the skin with adhesive pads.

When using FES techniques for spinal cord rehabilitation therapy, there is an extensive set-up procedure. The time required to position, attach, and secure the electrodes cause frustration for both the patients and practitioners due to electrode detachment, loss of electrode adhesion, and the additional equipment required to ensure the electrode is secured to the patient. The frustration is exacerbated by the additional time required to address these issues.

Any time delays during rehabilitation therapy result in less time allowed for participation in the therapy. This can lead to reduced therapeutic benefits as well as a decreased motivation to undertake the therapy.

The preferred embodiment reduces FES set-up time, allowing this saved time to be used for rehabilitation.

### SUMMARY OF THE INVENTION

According to one aspect of the present invention, there is provided an applicator for applying functional electrical stimulation (FES) to rehabilitate a person, the applicator including:
a surround for surrounding a portion of the person; and
one or more electrodes for locating within the surround.

The electrodes may be borne by the surround, when attaching the surround to the portion, to greatly reduce set-up time.

The applicator may include electrode fastening means for fastening the electrodes to the surround. The electrode fastening means may be releasable and/or position adjustable, preferably including a hook- and loop fastener. The fastener may be adhesive-backed.

The applicator may include surround fastening means for fastening the surround to itself. The surround fastening means may be releasable and/or tightness adjustable, preferably including a hook- and loop fastener.

The electrodes may include a gel coating, and a protector for protecting the gel coating. The protector may include a sheet, preferably of plastics material.

Each electrode may include a pad, and a conductor extending from the pad. The conductor may terminate in a connector or adaptor. The connector or adaptor may be located external the surround. The connector or adaptor may include a light emitting diode (LED).

The surround may include a resilient sheet, preferably a neoprene sheet. The portion may be a limb (e.g. leg). The surround may be shaped to fit at least part (e.g. upper part, lower part) of the limb. The applicator may include a garment. The garment may be a sleeve or collar.

The applicator may include a processor or computer borne by the surround.

According to another aspect of the present invention, there is provided a rehabilitation method involving:
surrounding a portion of a person with a surround; and
applying functional electrical stimulation (FES) to the person using one or more electrodes located within the surround.

Any of the features described herein can be combined in any combination with any one or more of the other features described herein within the scope of the invention.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 shows two open (FES) applicators in accordance with an embodiment of the present invention;

FIG. 2 shows one of the FES applicators of FIG. 1 applied to a patient; and

FIGS. 3A and 3B show the attachment of the electrodes to one of the FES applicators of FIG. 1.

### DETAILED DESCRIPTION OF PREFERRED EMBODIMENTS

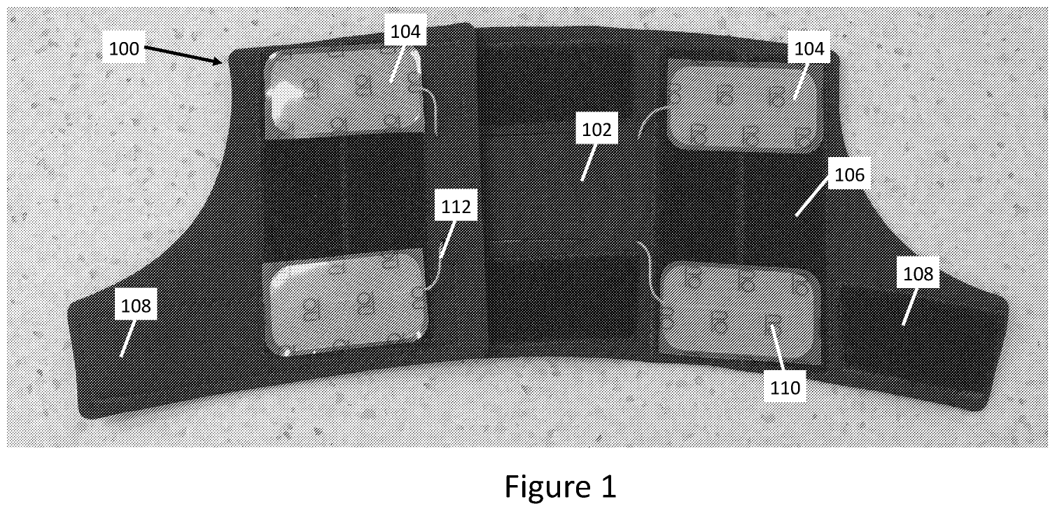

According to an embodiment of the present invention, there is provided an applicator 100 as shown in FIG. 1. The applicator 100 is suitable for applying functional electrical stimulation (FES) to rehabilitate a patient.

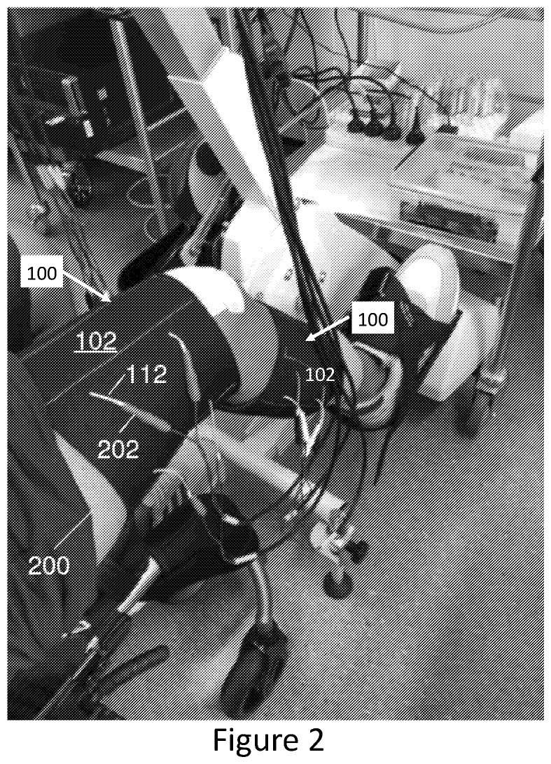

The applicator 100 includes a fabric surround 102 for surrounding a limb 200 (i.e. portion) of the patient (FIG. 2). The surround 102 includes a resilient neoprene sheet and forms a wearable sleeve garment. The sheet is shaped to fit part (e.g. upper part, lower part, etc.) of the limb 200 (e.g. leg).

Furthermore, the applicator 100 includes four electrodes 104, arranged in vertical pairs, for locating within the closed surround 102. The electrodes 104 are borne by the surround 102, when attaching the surround 102 to the limb 200, to greatly reduce set-up time.

The applicator 100 includes electrode fastening means 106 for releasably fastening the electrodes 104 in the surround 102. In particular, the electrode fastening means 106 includes adhesive-backed hook- and loop fasteners (e.g. Velcro™) to facilitate electrode positional adjustment.

Similarly, the applicator 100 includes surround fastening means 108 for releasably fastening the surround 102 to itself. In particular, the surround fastening means 108 includes a pair of hook- and loop fasteners to facilitate surround tightening adjustment.

Each electrode 104 includes a broad pad 110, and an electrical cable (i.e. conductor) 112 extending from the pad 110. The cable 112 extends through an aperture in the surround 102 and terminates in a connector 202 or adaptor. An electrical lead from electrical rehabilitation equipment, such as the neuromuscular electrical stimulator unit, can be readily connected to the connector 202 by plugging.

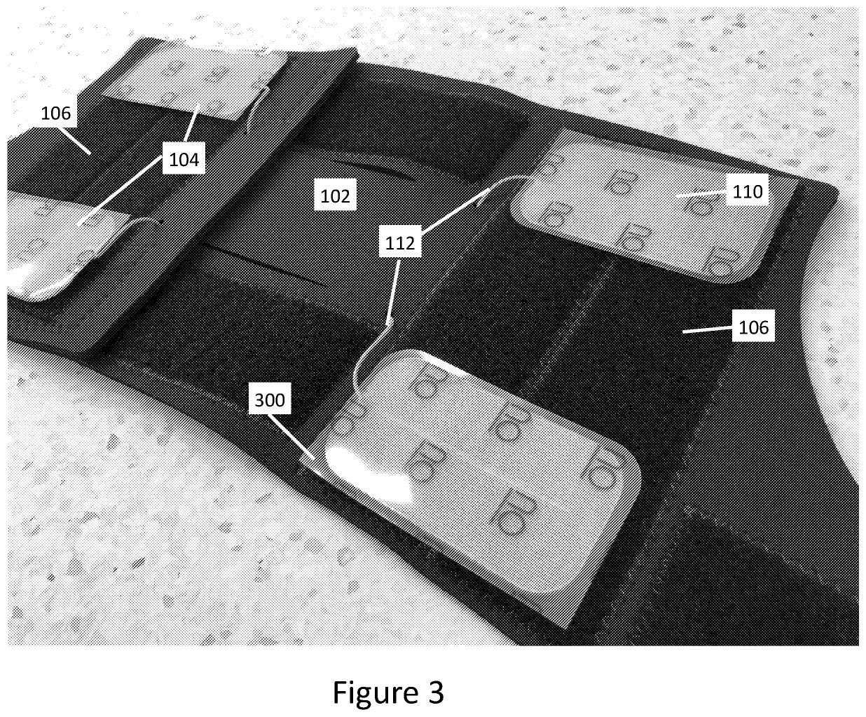

Turning to FIG. 3, the electrodes 104 include a hydro-gel surface coating to improve coupling with the skin of the limb 200, and a removable clear-plastic sheet protector 300 for protecting the gel coating.

A method of rehabilitation using the applicator is now briefly described.

Initially as shown in FIG. 3, the electrodes 104 are fastened at desirable locations on the open surround 102 using the electrode fastening means 106. The protector 300 is removed to expose the surface gel, and the electrodes 104 are positioned on the limb 200. The connectors 202 are pushed through the apertures in the surround 102.

Next, as shown in FIG. 2, ends of the surround 102 are fastened together with the surround fastening means 108 so that the surround 102 snugly surrounds the limb 200 with the electrodes 104 coupled to the patient's skin.

Next, electrical leads from electrical rehabilitation equipment are readily plugged into the external connectors 202. The electrical rehabilitation equipment applies FES to the person via the electrodes 104 located within the closed surround 102.

After rehabilitation, the surround 102 can be removed by separating the surround fastening means 108. The electrodes 104 borne by the surround 102 can be left in place, so they are correctly configured for the next rehabilitation treatment.

The applicator 100 can be secured to a patient in 2-3 minutes with the electrodes 104 in place, compared to the 10-15 minutes typically needed when the electrodes 104 are individually placed on the patient. The electrodes 104 can also be easily removed from the surround 102 and replaced as needed to allow continuous use of the applicator 100.

Although similar technologies currently exist, they do not meet the needs as met by the applicator 100. Existing electrodes are either dry electrodes, which require the use of gel to be applied to the legs of the user prior to therapy beginning, or snap-on electrodes which tend to “snap-off”, leading to increased setup and clean-up times.

The applicator 100 uses heavy-duty electrodes 104 allowing use in FES applications, which require higher stimulation for patients with spinal cord injuries (SCI), multiple sclerosis (MS), or stroke rehabilitation therapies, to name a few. The applicator 100 is also style agnostic, allowing the rehabilitation practitioner to use their preferred style of electrode 104.

The applicator 100 drastically reduces the set-up procedure for rehabilitation therapy.

The advantages of utilising the applicator 100 include:
Approx. 80% reduction in patient set-up time, with four electrodes 104 applied at once
Ability to prepare the electrodes 104 prior to the therapy session.
Ability to easily reposition electrodes 104 using hook-and-loop attachments of the electrode fastening means 106
Ability to wash applicator 100 (laser engraved graphics/instructions will not deteriorate).
Ability to reuse the applicator 100 without having to reattach the electrodes 104
Ability to use a single applicator size for multiple patients (neoprene material allows for variability in garment sizing), with the surround fastening means 108 providing for snug tightening of the applicator 100 to ensure good electrode contact even when adhesion properties deteriorate
Easy attachment of applicator 100 to patients (no need to remove shoes or clothing)
Efficient connection and routing of electrode cables 112

A person skilled in the art will appreciate that many embodiments and variations can be made without departing from the ambit of the present invention.

In one embodiment, the electrodes 104 are first applied to the patient, and then the separate surround 102 is applied to overlay the electrodes 104.

The applicator 100 can include an onboard processor or computer borne by the surround 102 and for processing signals from the electrodes 104 or for sending signals to the electrodes 104. The onboard processor performs at least some of the functions of the neuromuscular electrical stimulator.

Each connector 202 can include a light emitting diode (LED) forming part of a cable management system which makes locating electrode conductors 112 quick and easy, allowing the conductors 112 to be efficiently routed and connected. The LED lighting is used as a safety check for practitioners to see if the correct cables 112 are running to the correct electrode 104.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

1. An applicator for applying functional electrical stimulation (FES) to rehabilitate a person, the applicator including:
a surround for surrounding a portion of the person; and
one or more electrodes for locating within the surround.

2. An applicator as claimed in claim 1, wherein the electrodes are borne by the surround, when attaching the surround to the portion, to greatly reduce set-up time.

3. An applicator as claimed in claim 1, including electrode fastening means for fastening the electrodes to the surround.

4. An applicator as claimed in claim 3, wherein the electrode fastening means is releasable and/or position adjustable.

5. An applicator as claimed in claim 3, wherein the electrode fastening means includes a hook- and loop fastener.

6. An applicator as claimed in claim 5, wherein the fastener is adhesive-backed.

7. An applicator as claimed in claim 1, including surround fastening means for fastening the surround to itself.

8. An applicator as claimed in claim 7, wherein the surround fastening means is releasable and/or tightness adjustable.

9. An applicator as claimed in claim 7, wherein the surround fastening means includes a hook- and loop fastener.

10. An applicator as claimed in claim 1, wherein the electrodes include a gel coating, and a protector for protecting the gel coating.

11. An applicator as claimed in claim 10, wherein the protector includes a sheet.

12. An applicator as claimed in claim 11, wherein the sheet includes plastics material.

13. An applicator as claimed in claim 1, wherein each electrode includes a pad, and a conductor extending from the pad.

14. An applicator as claimed in claim 13, wherein the conductor terminates in a connector or adaptor.

15. An applicator as claimed in claim 14, wherein the connector or adaptor is located external the surround.

16. An applicator as claimed in claim 14, wherein the connector or adaptor includes a light emitting diode (LED).

17. An applicator as claimed in claim 1, wherein the surround includes a resilient sheet, preferably a neoprene sheet.

18. An applicator as claimed in claim 1, wherein the portion includes a limb, and the surround is shaped to fit at least part of the limb.

19. An applicator as claimed in claim 1, wherein the applicator includes a garment.

20. An applicator as claimed in claim 1, including a processor or computer borne by the surround.

21. A rehabilitation method involving:
surrounding a portion of a person with a surround; and
applying functional electrical stimulation (FES) to the person using one or more electrodes located within the surround.
